(19) United States
(12) Patent Application Publication
(10) Pub. No.: US 2021/0327066 A1
(43) Pub. Date: Oct. 21, 2021

(54) APPARATUS AND METHOD FOR DETERMINING MUSCULOSKELETAL DISEASE

(71) Applicant: ELECTRONICS AND TELECOMMUNICATIONS RESEARCH INSTITUTE, Daejeon (KR)

(72) Inventors: Daehwan KIM, Sejong-si (KR); Yong Wan KIM, Daejeon (KR); Ki Suk LEE, Daejeon (KR)

(73) Assignee: ELECTRONICS AND TELECOMMUNICATIONS RESEARCH INSTITUTE, Daejeon (KR)

(21) Appl. No.: 17/235,192

(22) Filed: Apr. 20, 2021

(30) Foreign Application Priority Data
Apr. 21, 2020 (KR) 10-2020-0047910

(51) Int. Cl. G06T 7/00; G06T 7/70; G06T 7/20; G16H 50/20; G16H 50/50; G06N 3/04; G06N 3/08

(52) U.S. Cl. CPC G06T 7/0014; G06T 7/70; G06T 7/20; G16H 50/20; G16H 50/50; G06T 2207/20084; G06N 3/0445; G06N 3/088; G06T 2207/20081; G06T 2207/30008; G06N 3/0454

(57) ABSTRACT

An apparatus for determining a musculoskeletal disease may be disclosed. The apparatus may include a motion protocol learning unit configured to generate a first motion protocol used to determine a musculoskeletal disease in advance through learning, a motion protocol recognition model unit configured to generate a motion protocol recognition model for determining a musculoskeletal disease by using information of the first motion protocol, a body pose estimator configured to receive a user image to be recognized and estimate a body pose from the user image, and a disease classification and prediction unit configured to determine a musculoskeletal disease by matching the body pose and the motion protocol recognition model.

### CROSS-REFERENCE TO RELATED APPLICATION

This application claims priority to and the benefit of Korean Patent Application No. 10-2020-0047910 filed in the Korean Intellectual Property Office on Apr. 21, 2020, the entire contents of which are incorporated herein by reference.

### BACKGROUND OF THE INVENTION

### (a) Field of the Invention

The following description relates to an apparatus and method for determining musculoskeletal disease.

### (b) Description of the Related Art

Recently, technologies for recognizing and interacting with postures or motions of human body through image analysis have emerged. Microsoft Kinect's human-body skeleton detection technology was developed around 2010, and since then, many similar technologies have been announced that have improved recognition performance. Since such a posture recognition technology has a merit capable of analyzing motion without wearing wearable equipment, it is applied to various fields such as personal training, entertainment-type exercise, posture correction, function games, robots, and shopping. Meanwhile, as the performance of posture recognition technologies is getting higher and higher, their use is increasing in fields such as health, disease analysis, and virtual environment interaction that require high accuracy. However, due to optical capture method, these technologies are somewhat problematic in fields such as posture measurement and health disease prediction, which are heavily affected by fine movements.

Due to the merit of image-based motion recognition technology that does not require wearing complex equipment, studies are being attempted to use it for disease classification and prediction. Existing disease analysis methods predict disease by measuring minute angular movements of each joint. However, due to the performance limitation of image-based posture recognition, it may not be suitable for disease analysis methods based on fine movements. Accordingly, there is a need for a disease analysis method using elements other than a disease analysis method based on fine movements.

The above information disclosed in this Background section is only for enhancement of understanding of the background of the invention, and therefore it may contain information that does not form the prior art that is already known in this country to a person of ordinary skill in the art.

### SUMMARY OF THE INVENTION

At least one of the embodiments may provide an apparatus and method for more accurately and easily determining musculoskeletal diseases based on an image.

In one aspect, an apparatus for determining a musculoskeletal disease may be provided. The apparatus may include a motion protocol learning unit configured to generate a first motion protocol used to determine a musculoskeletal disease in advance through learning, a motion protocol recognition model unit configured to generate a motion protocol recognition model for determining a musculoskeletal disease by using information of the first motion protocol, a body pose estimator configured to receive a user image to be recognized and estimate a body pose from the user image, and a disease classification and prediction unit configured to determine a musculoskeletal disease by matching the body pose and the motion protocol recognition model.

The first motion protocol may be a motion sequence capable of determining a musculoskeletal disease among human motions.

The information of the first motion protocol may include an entire list of the first motion protocol and skeleton sequence information on the first motion protocol.

The apparatus may further include motion a protocol generator configured to automatically generate a second motion protocol, and the motion protocol learning unit may learn the second motion protocol and generate the first motion protocol suitable for determining a musculoskeletal disease.

The motion protocol generator may include a random body pose generator configured to generate a human body pose that can be combined using data of an available range of movements for each human body joint, and a random human body pose time series generator configured to generate the second motion protocol by connecting the human body pose in time series.

The motion protocol generator may generate the second motion protocol by using data of an available range of movements for each human body joint and a third motion protocol defined in advance.

The motion protocol generator may generate the second motion protocol through a generative adversarial network (GAN) and a recurrent neural network (RNN).

The motion protocol learning unit may include an identification unit configured to classify a third motion protocol that can be performed by a person by learning the second motion protocol and a fourth motion protocol defined in advance, and a recognition model generator configured to generate the first motion protocol by learning the third motion protocol.

The disease classification and prediction unit may include a disease classifier configured to determine a musculoskeletal disease by matching the body pose and the motion protocol recognition model, and a disease predictor configured to calculate the determined result of the disease classifier in probability form.

In another aspect, a method for determining a musculoskeletal disease of a user to be recognized by an apparatus may be provided.

The method may include generating a first motion protocol used to determine whether there is a musculoskeletal disease through learning, generating a motion protocol recognition model for classifying or predicting a musculoskeletal disease by using information of the first motion protocol, estimating a body pose from an image of the user, and determining a musculoskeletal disease by matching the body pose and the motion protocol recognition model.

The first motion protocol may be a motion sequence suitable for determining a musculoskeletal disease among human motions.

The information of the first motion protocol may include an entire list of the first motion protocol and skeleton sequence information on the first motion protocol.

The method may further include generating a human body pose that can be combined using data of an available range of movements for each human body joint, and generating the second motion protocol by connecting the human body pose in time series, and the generating of the first motion protocol may include generating the first motion protocol by learning the second motion protocol.

The method may further include generating a second motion protocol by using data of an available range of movements for each human body joint and a third motion protocol defined in advance, and the generating of the first motion protocol may include generating the first motion protocol by learning the second motion protocol.

The generating of the first motion protocol may include classifying a third motion protocol that can be performed by a person by learning the second motion protocol and a fourth motion protocol defined in advance, and generating the first motion protocol by learning the third motion protocol.

In another aspect, a method for determining a musculoskeletal disease of a user to be recognized by an apparatus may be provided. The method may include generating a first motion protocol, which is a motion capable of determine a musculoskeletal disease among human motions, through learning, generating a motion protocol recognition model by using the first motion protocol, estimating a body pose from an image of the user, and determining a musculoskeletal disease by using the body pose and the motion protocol recognition model.

The method may further include generating a second motion protocol by learning, and the generating of the first motion protocol may include the first motion protocol suitable for determining a musculoskeletal disease by learning the second motion protocol.

Such an embodiment can more accurately and easily determine a musculoskeletal disease by using a motion protocol.

### BRIEF DESCRIPTION OF THE DRAWINGS

FIG. 1 is a block diagram showing an apparatus for determining a musculoskeletal disease, according to one embodiment.

FIG. 2A is a bock diagram showing a motion protocol generator, according to one embodiment.

FIG. 2B is a block diagram showing a motion protocol generator, according to another embodiment.

FIG. 3 is a block diagram showing a motion protocol learning unit, according to one embodiment.

FIG. 4 is a block diagram showing a motion protocol recognition model unit, according to one embodiment.

FIG. 5 is a block diagram showing a body pose estimator, according to one embodiment.

FIG. 6 is a block diagram showing a disease classification and prediction unit, according to one embodiment.

FIG. 7 is a diagram showing a method for classifying and predicting a disease, according to one embodiment.

FIG. 8 is a diagram showing a computer system, according to one embodiment.

### DETAILED DESCRIPTION OF THE EMBODIMENTS

The following detailed description is provided to assist the reader in gaining a comprehensive understanding of the methods, apparatuses, and/or systems described herein. However, various changes, modifications, and equivalents of the methods, apparatuses, and/or systems described herein will be apparent after understanding of the disclosure of this application. For example, the sequences of operations described herein are merely examples, and are not limited to those set forth herein, but may be changed as will be apparent after understanding of the disclosure of this application, with the exception of operations necessarily occurring in a certain order. Also, descriptions of features that are known in the art may be omitted for increased clarity and conciseness. The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after understanding of the disclosure of this application. Throughout the specification, when an element such as a layer, region, or substrate is described as being “on” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween. As used herein, the term “and/or” includes any one and any combination of any two or more of the associated listed items. Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples. Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly. The terminology used herein is for describing various examples only, and is not to be used to limit the disclosure. The articles “a,” “an” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof. The features of the examples described herein may be combined in various ways as will be apparent after an understanding of the disclosure of this application. Further, although the examples described herein have a variety of configurations, other configurations are possible as will be apparent after an understanding of the disclosure of this application. In addition, unless explicitly described to the contrary, the word “comprise” and variations such as “comprises” or “comprising” will be understood to imply the inclusion of stated elements but not the exclusion of any other elements.

The apparatus and method for determining a musculoskeletal disease according to one embodiment may generate a motion protocol model suitable for musculoskeletal disease analysis, estimate the posture of a person performing the motion protocol in an image, and analyze the relationship between the estimated posture of the person and the motion protocol model. Through this, it is possible to classify and predict a musculoskeletal disease. Hereinafter, an apparatus and method for determining a musculoskeletal disease according to at least one embodiment will be described in detail.

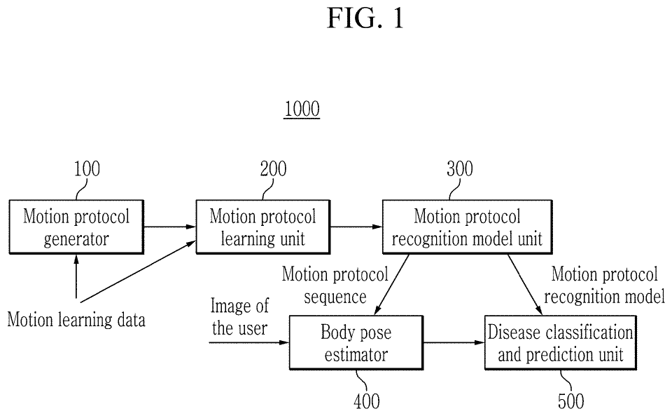

FIG. 1 is a block diagram showing an apparatus for determining a musculoskeletal disease, according to one embodiment.

As shown in FIG. 1, the apparatus 1000 according to one embodiment includes a motion protocol generator 100, a motion protocol learning unit 200, a motion protocol recognition model unit 300, a body pose estimator 400, and a disease classification and prediction unit 500.

The motion protocol generator 100 automatically generates a predetermined motion protocol. The motion protocol learning unit 200 learns the motion protocol generated by the motion protocol generator 100 to generate motion protocols most suitable for classification and recognition. The operation protocol recognition model unit 300 generates a motion protocol sequence and a motion protocol recognition model by using the motion protocols generated by the motion protocol learning unit 200. The body pose estimator 400 estimates a body pose of the user by using the image of the user to be recognized. In addition, the disease classification and prediction unit 500 classifies and predicts a musculoskeletal disease by matching the body pose estimated by the body pose estimator 400 and the motion protocol recognition model generated by the motion protocol recognition model unit 300 with each other.

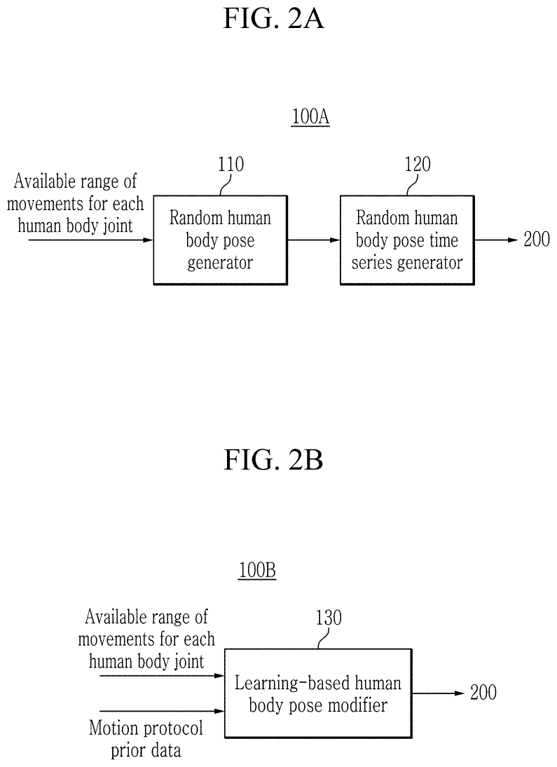

First, a detailed configuration and operation of the motion protocol generator 100 according to one embodiment will be described with reference to FIGS. 2A and 2B.

The motion protocol refers to a pre-generated motion sequence to classify diseased and non-diseases. That is, motion protocols are time-series motion types that can cause the postures of the diseased and non-disease to react differently even if they perform the same motion. For example, the motion protocol is a motion sequence in which the shape and sequence of motions such as lunges, squats, planks, etc. are defined. In other words, the motion protocol is not a simple series of motions, but refers to motions that induce postures in which the diseased and non-disease have to show different motions due to the musculoskeletal problem of a specific joint. However, since it is not known which operations can provide such an effect, in one embodiment, two methods (FIG. 2A, FIG. 2B) are presented to automatically generate a motion protocol.

FIG. 2A is a block diagram showing a motion protocol generator 100A, according to one embodiment. The motion protocol generator 100A according to one embodiment generates motions at random.

As shown in FIG. 2A, the motion protocol generator 100A according to one embodiment includes a random human body pose generator 110 and a random human body pose time series generator 120.

The random human body pose generator 110 receives data of an available range of movements for each human body joint, and generates a human body pose that combines possible movements of all joints using the received data (data of the available range of movements for each human body joint). Here, the human body pose refers to a form of motion of the human body at a certain point in time.

For example, the random human body pose generator 110 generates a human body pose at a moment by using data of an available range of movements for each human body joint.

The random human body pose time series generator 120 generates a motion protocol that is a continuous motion by connecting the human body poses generated by the random human body pose generator 110 in time series. Since the motion protocol is a continuous motion, the random human body pose time series generator 120 creates a motion protocol, which is a single movement, by connecting the poses at each point in time generated by the random human body pose generator 110. Here, the motion is a sequence connecting human body poses. That is, the random human body pose time series generator 120 generates a pose in time series by considering the range of the previous frame motion in consideration of the continuation between motion frames.

Meanwhile, the motion protocol generator 100A generates a motion protocol using a Generative Adversarial Network (GAN) and a Recurrent Neural Network (RNN). The Generative Adversarial Network (GAN) can generate various motions, and the Recurrent Neural Network (RNN) can generate a natural motion protocol by continuously maintaining a person's pose within the available range of the human body. The specific operation method for the generative adversarial neural network and the recurrent neural network can be known to those of ordinary skill in the art, and a detailed description thereof will be omitted.

FIG. 2B is a block diagram showing a motion protocol generator 100B, according to another embodiment. The motion protocol generator 100B according to another embodiment regenerates other motions based on prior motions.

As shown in FIG. 2B, the motion protocol generator 100B includes a learning-based human body pose modifier 130.

The learning-based human body pose modifier 130 receives data of an available range of movements for each human body joint and motion protocol prior data (motion learning data), and generates motion protocols by creating and transforming various motions using these two sets of input data. Here, the motion protocol prior data is data on previously given motions. The learning-based human body pose modifier 130 may also generate a motion protocol using a Generative Adversarial Network (GAN) and a Recurrent Neural Network (RNN), which are algorithms such as the motion protocol generator 100A according to the one embodiment.

However, since the learning-based human body pose modifier 130 does not randomly generate movements (action protocol), it is possible to generate movements (motion protocol) of a correct shape within a short time. That is, the learning-based human body pose modifier 130 can quickly and accurately generate a motion (motion protocol) without the need to extensively search for a dimension of joint position data representing a motion.

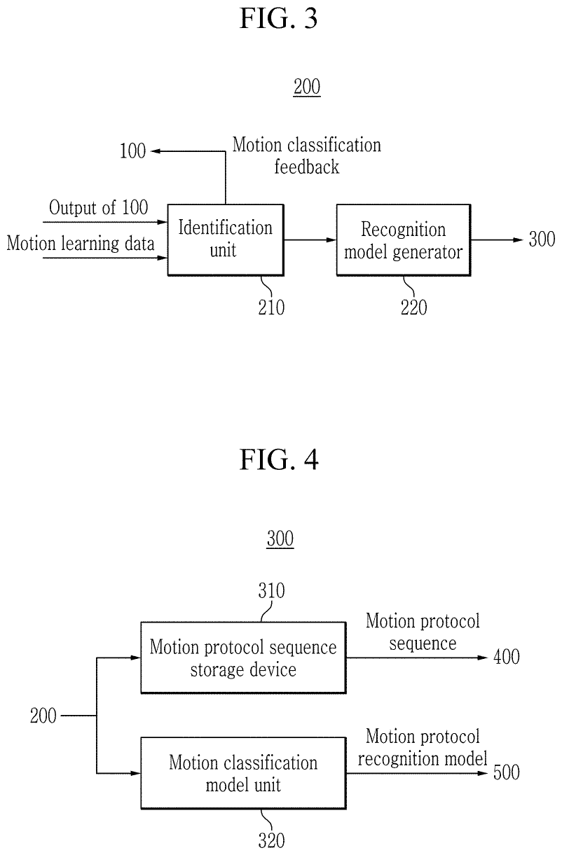

FIG. 3 is a block diagram showing a motion protocol learning unit 200, according to one embodiment. The motion protocol learning unit 200 according to one embodiment learns the motion protocol generated by the motion protocol generator 100 to generate motion protocols most suitable for classification and recognition.

As shown in FIG. 3. The motion protocol learning unit 200 according to one embodiment includes an identification unit 210 and a recognition model generator 220.

The identification unit 210 receives the motion protocol generated by the motion protocol generator 100 and the motion learning data (the pre-determined motion protocol prior data), and classifies a motion protocol that can be performed by a person by repeatedly comparing the two received sets of data. The identification unit 210 performs learning and identification by applying a Generative Adversarial Network (GAN) using the two received sets of data (the motion protocol generated by the motion protocol generator 100 and the motion learning data).

Meanwhile, the identification unit 210 feeds back the result of comparing the two sets of data (motion identification result) to the motion protocol generator 100, and the motion protocol generator 100 modifies and regenerates the previously generated motion protocol using the information received from the feedback. In addition, the identification unit 210 repeats the operation by receiving the corrected and regenerated motion protocol again. This process may be repeated to a level at which the identification unit 210 does not distinguish between the two sets of data (the motion protocol generated by the motion protocol generator 100 and the motion learning data). Through this, the identification unit 210 may generate a motion that a person can perform. The motion protocol output by the motion protocol generator 100 may be a motion that a person cannot actually perform. These motions are practically meaningless, even if they can classify diseases well. In order to solve this part, the identification unit 210 may classify and generate a motion protocol that can be performed by a person by repeatedly comparing the two sets of data.

The recognition model generator 220 generates a motion protocol capable of distinguishing (recognizing) diseases well by using the motion protocol classified by the identification unit 210. That is, the recognition model generator 220 finally generates a motion protocol having a form that is capable of classifying diseases well. Here, the recognition model generator 220 may generate a motion protocol capable of distinguishing diseases well by using a Recurrent Neural Network (RNN) or a similar algorithm. Meanwhile, the recognition model generator 220 outputs information on the generated motion protocol to the motion protocol recognition model unit 300. Here, the information on the motion protocol output from the recognition model generator 220 includes an entire list of motion protocols and skeleton sequence information for each motion protocol. Hereinafter, the skeleton sequence information for the motion protocol is referred to as “motion protocol sequence”.

FIG. 4 is a block diagram showing a motion protocol recognition model unit 300, according to one embodiment.

As shown in FIG. 4, the motion protocol recognition model unit includes a motion protocol sequence storage device 310 and a motion classification model unit 320.

The motion protocol sequence storage device 310 stores information on the motion protocol output from the motion protocol learning unit 200. That is, the motion protocol sequence storage device 310 stores the entire list of motion protocols and the motion protocol sequence for each operation protocol output from the operation protocol learning unit 200. The motion protocol sequence stored in the motion protocol sequence storage device 310 may be used in the body pose estimator 400.

The motion classification model unit 320 generates and stores a motion protocol recognition mod& capable of classifying and predicting whether a person has a disease by using information on the motion protocol output from the motion protocol learning unit 200. Here, the motion protocol recognition model refers to the order or structural information of features and algorithms extracted from information on the motion protocol. For example, the recognition model extracts only the edge information of the video image and then performs recognition with the edge image. Alternatively, in a deep learning algorithm, the recognition model represents the shape and structure of the layers that make up a neural network, such as VGG-19 and Google Net.

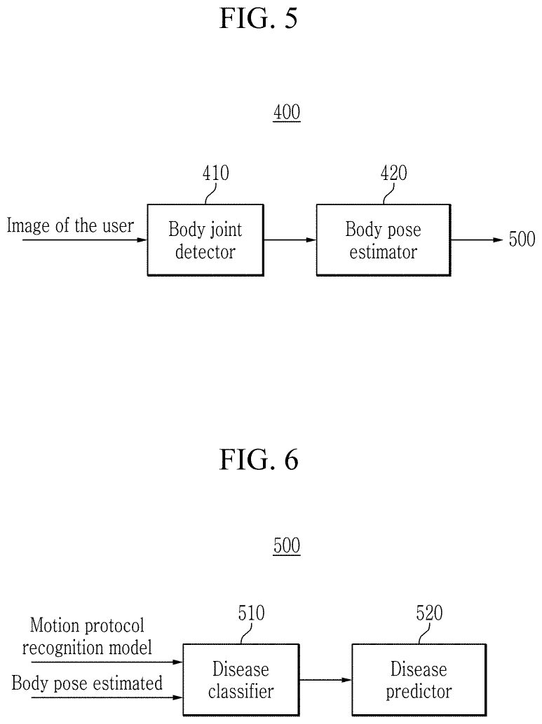

FIG. 5 is a block diagram showing a body pose estimator 400, according to one embodiment. The body pose estimator 400 according to one embodiment estimates a body pose by using an image of a user (a disease classification target) who is a recognition target.

As shown in FIG. 5, the body pose estimator 400 according to one embodiment includes a body joint detector 410 and a body pose estimator 420.

The user to be recognized actually performs the motion protocol described above, and the body joint detector 410 detects a joint in the user's body using an image of the user performing the motion protocol. That is, the user to be recognized actually performs the motion protocol described above in front of the camera, and the body joint detector 410 detects the joint from the image of the user taking the motion protocol. Here, the image of the user may be a 3D image and may be acquired through a camera. The body joint detector 410 acquires information on 2D or 3D positions such as head, shoulder, neck, knee, and foot, which are important joint positions of a person in the user's image. Various algorithms such as Kinect and Openpose may be used as a method of detecting a body joint, and a detailed description thereof will be omitted since such a method can be known to those of ordinary skill in the art.

The body pose estimating unit 420 estimates the user's body pose using the body joint detected by the body joint detector 410. The body pose estimator 420 generates a skeleton by connecting body joints detected by the body joint detector 410, and estimates a body pose of the user based on the generated skeleton. Meanwhile, a method of estimating a body pose can be known to those of ordinary skill in the art, and thus a detailed description thereof will be omitted. The body pose estimator 420 outputs the result of he estimated body pose to the disease classification and prediction unit 500.

Since the apparatus for determining a musculoskeletal disease according to one embodiment determines a musculoskeletal disease using an operation protocol, it is possible to more accurately and easily determine a musculoskeletal disease even if the disease is determined based on an image.

FIG. 6 is a block diagram showing a disease classification and prediction unit 500, according to one embodiment. The disease classification and prediction unit 500 according to one embodiment classifies and predicts musculoskeletal disease by matching the body pose estimated by the body pose estimator 400 and the motion protocol recognition model generated by the motion protocol recognition model unit 300 with each other.

As shown in FIG. 6, the disease classification and prediction unit 500 according to one embodiment includes a disease classifier 510 and a disease predictor 520.

The disease classifier 510 receives the result of the body pose estimated by the body pose estimator 400 and the motion protocol recognition model generated by the motion protocol recognition mod& unit 300, and determines whether there is a disease (musculoskeletal disease) by using the two received sets of data. That is, the disease classifier 510 determines whether the disease is a disease by determining whether the result of the estimated body pose matches the motion protocol recognition model.

In addition, the disease predictor 520 calculates and outputs the probability of a disease determined by the disease classifier 510 in a probability form.

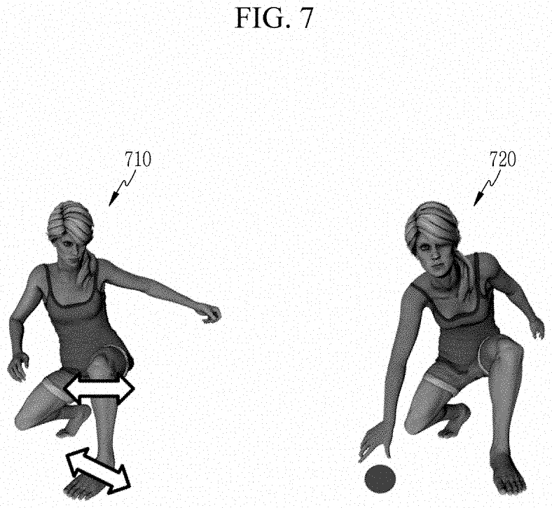

Here, the method of classifying and predicting a disease by the disease classification and prediction unit 500 may be largely used in two ways. FIG. 7 is a diagram showing a method for classifying and predicting a disease, according to one embodiment.

Referring to 710 of FIG. 7, the first method is a method of analyzing the relationship between the characteristics (angle, position, degree of change, direction, etc.) of body joints. This is a method of analyzing feature values when an action is taken rather than completing the overall action.

Referring to 720 of FIG. 7, the second method is a motion mission completion type. Since there is a point that the accuracy of joint tracking is not high, this method is a method of determining disease classification based on a result of whether the motion has been completed when the entire motion sequence is performed.

Among these two methods, it can be used alternatively or mixedly.

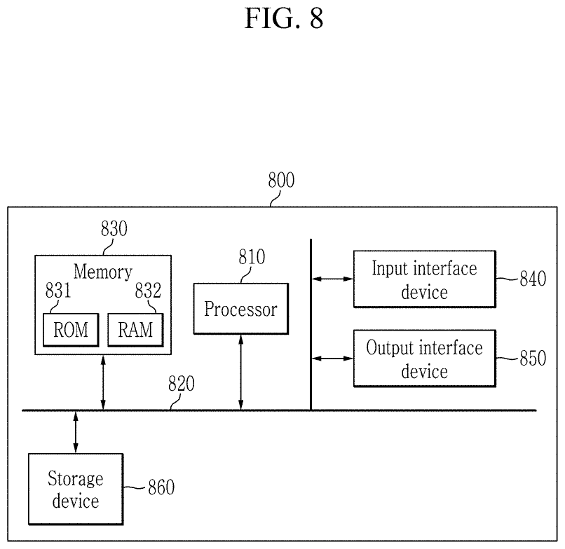

FIG. 8 is a diagram showing a computer system 800, according to one embodiment,

The apparatus 1000 for determining a musculoskeletal disease according to one embodiment may be implemented in the computer system 800 of FIG. 8. Each component of the apparatus 1000 can also be implemented in the computer system 800 of FIG. 8.

The computer system 800 can include at least one of a processor 810, a memory 830, an input interface device 840, an output interface device 850, and a storage device 860, that communicate via a bus 820.

The processor 810 can be a central processing (CPU) or a semiconductor device that executes instructions stored in the memory 830 or the storage device 860. The processor 810 can be configured to implement the functions and methods described in FIG. 1 to FIG. 7.

The memory 830 and the storage device 860 can include various forms of volatile or non-volatile storage media. For example, the memory 830 can include a read only memory (ROM) 831 and a random access memory (RAM) 832

In one embodiment, the memory 830 may be located inside or outside the processor 810, and the memory 830 can be coupled to the processor 810 through various already-known means.

While this disclosure includes specific examples, it will be apparent after understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or theft equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for determining a musculoskeletal disease, the apparatus comprising:
a motion protocol learning unit configured to generate a first motion protocol used to determine a musculoskeletal disease in advance through learning;
a motion protocol recognition model unit configured to generate a motion protocol recognition model for determining a musculoskeletal disease by using information of the first motion protocol;
a body pose estimator configured to receive a user image to be recognized and estimate a body pose from the user image; and
a disease classification and prediction unit configured to determine a musculoskeletal disease by matching the body pose and the motion protocol recognition model.

2. The apparatus of claim 1, wherein
the first motion protocol is a motion sequence capable of determining a musculoskeletal disease among human motions.

3. The apparatus of claim 1, wherein
the information of the first motion protocol includes an entire list of the first motion protocol and skeleton sequence information on the first motion protocol.

4. Th apparatus of claim 1, further comprising
a motion protocol generator configured to automatically generate a second motion protocol,
wherein the motion protocol learning unit learns the second motion protocol and generates the first motion protocol suitable for determining a musculoskeletal disease.

5. The apparatus of claim 4, wherein
the motion protocol generator includes:
a random body pose generator configured to generate a human body pose that can be combined using data of an available range of movements for each human body joint; and
a random human body pose time series generator configured to generate the second motion protocol by connecting the human body pose in time series.

6. The apparatus of claim 4, wherein
the motion protocol generator generates the second motion protocol by using data of an available range of movements for each human body joint and a third motion protocol defined in advance.

7. The apparatus of claim 4, wherein
the motion protocol generator generates the second motion protocol through a generative adversarial network (GAN) and a recurrent neural network (RNN).

8. The apparatus of claim 4, wherein
the motion protocol learning unit includes:
an identification unit configured to classify a third motion protocol that can be performed by a person by learning the second motion protocol and a fourth motion protocol defined in advance; and
a recognition model generator configured to generate the first motion protocol by learning the third motion protocol.

9. The apparatus of claim 1, wherein
the disease classification and prediction unit includes:
a disease classifier configured to determine a musculoskeletal disease by matching the body pose and the motion protocol recognition model; and
a disease predictor configured to calculate the determined result of the disease classifier in probability form.

10. A method for determining a musculoskeletal disease of a user to be recognized, by an apparatus, the method comprising:
generating a first motion protocol used to determine whether there is a musculoskeletal disease through learning;
generating a motion protocol recognition model for classifying or predicting a musculoskeletal disease by using information of the first motion protocol;
estimating a body pose from an image of the user; and
determining a musculoskeletal disease by matching the body pose and the motion protocol recognition model.

11. The method of claim 10, wherein
the first motion protocol is a motion sequence suitable for determining a musculoskeletal disease among human motions.

12. The method of claim 10, wherein
the information of the first motion protocol includes an entire list of the first motion protocol and skeleton sequence information on the first motion protocol.

13. The method of claim 10, further comprising:
generating a human body pose that can be combined using data of an available range of movements for each human body joint; and
generating the second motion protocol by connecting the human body pose in time series,
wherein the generating of the first motion protocol includes generating the first motion protocol by learning the second motion protocol.

14. The method of claim 10, comprising
generating a second motion protocol by using data of an available range of movements for each human body joint and a third motion protocol defined in advance, and
the generating of the first motion protocol includes generating the first motion protocol by learning the second motion protocol.

15. The method of claim 13, wherein
the generating of the first motion protocol includes:
classifying a third motion protocol that can be performed by a person by learning the second motion protocol and a fourth motion protocol defined in advance; and
generating the first motion protocol by learning the third motion protocol.

16. A method for determining a musculoskeletal disease of a user to be recognized, by an apparatus, the method comprising:
generating a first motion protocol, which is a motion capable of determine a musculoskeletal disease among human motions, through learning;
generating a motion protocol recognition model by using the first motion protocol;
estimating a body pose from an image of the user; and
determining a musculoskeletal disease by using the body pose and the motion protocol recognition model.

17. The method of claim 16, further comprising
generating a second motion protocol by learning,
wherein the generating of the first motion protocol includes the first motion protocol suitable for determining a musculoskeletal disease by learning the second motion protocol.
